(19) United States
(12) Patent Application Publication
(10) Pub. No.: US 2015/0164414 A1
(43) Pub. Date: Jun. 18, 2015

(54) Recursive Real-time Determination of Glucose Forcing in a Diabetic patient for use in a Closed Loop Insulin Delivery system

(71) Applicant: Grant Matthews, Fort Wayne, IN (US)

(72) Inventor: Grant Matthews, Fort Wayne, IN (US)

(21) Appl. No.: 14/555,832

(22) Filed: Nov. 28, 2014

Related U.S. Application Data
(60) Provisional application No. 61/913,962, filed on Dec. 10, 2013.

(51) Int. Cl. A61B 5/00; A61B 5/145

(52) U.S. Cl. CPC A61B 5/4839; A61B 5/14532; A61B 5/7275; A61B 5/725; A61B 5/4866; A61B 5/7257; A61B 5/7225; A61B 5/0004; A61B 5/6801

(57) ABSTRACT

Presented is a computational system for predicting the blood glucose level to which a diabetic patient is being forced based solely on continuous glucose monitor (CGM) data that then allows optimum and safe calculation of a stabilizing dose to be applied by an insulin pump. This invention hence operates as part of a closed loop insulin delivery system. Included are recursive filters for estimating forthcoming blood glucose levels in real-time. Designed to match typically observed human blood glucose rates of change due to food digestion and insulin injection, these filters are two and three term exponential functions respectively. Such filters are applied to low pass filtered CGM data before being iteratively matched to the raw CGM data in order to yield greater confidence in the recursive predictions. All filters also have infinite response curves with monotonically decreasing amplitudes over time. The recursive and iterative process repeats with the arrival of further CGM measurements, allowing on-going calculation and delivery of optimum and safe insulin by an infusion pump in a close loop insulin delivery system.

TABLE 0.1
U.S. PATENT DOCUMENTS CITED

| | | |
|---|---|---|
| 6,554,800 B1 | April 2003 | Nezhadian & Orchard |
| 7,591,801 B2 | September 2009 | Brauker et al |
| 7,976,492 B2 | July 2011 | Brauker et al |
| 8,260,393 B2 | September 2012 | Kamath et al |
| 8,292,810 B2 | October 2012 | Goode et al |
| 8,346,338 B2 | January 2013 | Goode et al |
| 8,460,231 B2 | June 2013 | Brauker et al |
| 8,589,106 B2 | November 2013 | Engelhardt et al |
| 61/913,962 | December 2013 | Matthews |
| 8,690,820 B2 | April 2014 | Cinar & Oruklu |
| 8,784,370 B2 | July 2014 | Lebel & Starkweather |

### FIELD OF THE INVENTION

The present invention relates to the field of closed loop diabetic insulin control using a Continuous Glucose Monitoring (CGM) system and an insulin pump. More specifically, the present invention relates to using a recursive filter for real-time prediction of the blood glucose level toward which a diabetic patient is being forced, after food or insulin intake.

TABLE 0.2
OTHER REFERENCES CITED

| | | |
|---|---|---|
| Diabetes mellitus modeling and | Mathematical | F. Stahl & |
| short-term prediction based on | Biosciences 217, | R. Johansson |
| blood glucose measurements. | pp. 101-117 | 2009 |
| In-Flight Spectral | Journal of | Matthews |
| Characterization and | Atmospheric and | 2009 |
| Calibration Stability | Oceanic Technology | |
| Estimates for the Clouds and | 26(9) pp. 1685-1716 | |
| the Earth's Radiant Energy | | |
| System (CERES). | | |

### BACKGROUND OF THE INVENTION

The following background paragraph makes reference to FIG. 1(a) with its annotations numbered [1] to [5]. Like all systems, the blood glucose level in the human body responds with an infinite time impulse response function to an external forcing. Such forcing upwards can arise after food is broken down to chemical energy (sugar) by the stomach/digestive track[1]. Downward forcing typically occurs due to an amount of physical work being done by the body's muscles[2], which also requires the hormone insulin to mobilize or convert the blood sugar to energy[3]. In nondiabetics, a natural negative feedback mechanism[4] exists between the pancreas and liver that responds to these forcings as needed. For example an increasing blood sugar content will trigger a release of insulin from the pancreas beta cells, which allows the muscles to mobilize the sugar and convert [3] it into kinetic energy[2]. Typically a falling blood sugar will result in the release of glucagon also from the pancreas, which allows conversion of glycogen to glucose in the liver. This is then released into the blood to restore the glucose level[5], providing the extra energy needed for continued activity. In diabetics this mechanism has failed.

This background paragraph now makes reference to FIG. 1(b), with its annotations numbered [6] to [11]. In medical cases known as type 1 (or juvenile) diabetics, the pancreas organ no longer produces sufficient insulin or glucagon that triggers glucose creation within the liver[6]. Without the hormone insulin needed to create energy from sugar, the type 1 diabetic must typically inject artificially produced insulin[7] so their muscles can make use of the energy from food digestion. This then prevents hyper-glycemia, or glucose from building up in their blood supply, which is the primary cause of serious health problems in later life. Equally challenging are then problems due to the lack of negative feedback[6] in the opposite direction, that triggers conversion of glycogen to glucose in the liver for release into the blood supply. This means that if the patient injects too much artificial insulin, there is no restoring forcing mechanism to maintain blood sugar at levels needed for human function. This can result in hypo-glycemia, or low blood sugar with might lead to coma and death.

Type 2 (or adult onset) diabetics typically develop the condition later in life, which is where the body develops a resistance to, or general lack of, the insulin created by the pancreas. This can hence also be considered a perturbation to the body's blood glucose forcing and feedback system, but can often be controlled with use of oral medications. Like type 1 diabetics, such patients would also benefit from added feedback control (e.g. from a closed loop insulin delivery system shown in FIG. 1(b)). As before however, care is needed to ensure too much medication is not delivered because the pancreatic/liver restoration system can also be diminished[6] in these medical cases.

As stated the system is summarized by figure FIG. 1, which details the feedback mechanism that exists in the human body but breaks down in diabetics. The solution that is needed is a way to effectively and artificially re-create the natural system of FIG. 1(a), as shown in FIG. 1(b). This requires an algorithm[8] that takes data from a CGM [9] and predicts the blood sugar level to which the system is being forced from food digestion or insulin previously injected. This will allow a computer running the algorithm methodology developed herein[8] to calculate a further insulin dose[7] calibrated to the patient in question. This shall then restore the blood glucose level to the ideal range (between 80 and 120 mg/dl) in re-creation of the natural system of FIG. 1(a). It must also be designed to operate safely in a system where no automated sugar increasing restoring feedback is present (i.e. without an equivalent of the pancreas releasing glucagon to restore sugar levels from the liver[6]). Today it is possible to equip an insulin pump[10] with a separate glucose chamber[11] also for injection that would serve this purpose. However the invention must be designed to have a sufficiently sophisticated insulin predictive model, such that the sugar restoration system need only be used in the event of an emergency.

To do this, both a practical and effective time impulse response of the human blood glucose level and its changes after food and insulin intake must be developed. The time domain response must be of a nature that it can be matched to CGM data in real-time. This is so to allow a computer to make timely predictions of the final sugar level to which the patient is headed (i.e. in the absence of any additional food or insulin injections).

### OBJECTS OF THE INVENTION

It is an initial objective of this invention to design a mathematical representation of the human blood glucose time response to food and insulin intake (e.g. based on FIG. 2(a) taken from Stahl & Johansson (2009)). This must operate with minimal unknown mathematical variables such that it can be matched in real-time to data received from a CGM device. The matching must allow for different food types with various glucose absorption/conversion rates (e.g. carbo-hydrates vs. protein). Then multiple types of insulin (both fast and slow acting) must be considered, along with how the patient blood sugar will respond in terms of amplitude and time (noting also possible variance depending on environment and exercise etc). To allow the model to be adaptive it will be represented using a recursive filter, which will ease fast matching and prediction of the future state to which the patient is headed.

Still further, other objects and advantages of the invention with respect to modeling and adapting to the human body will be apparent from the specification and drawings.

### SUMMARY OF THE INVENTION

Time and environment dependant varying responses of the human blood glucose system to food, insulin and exercise must be adapted to. This will be done with a recursive filter based on a mathematical model of time response. Its construction shall use simple exponential functions to match the natures displayed in FIG. 2(a).

The invention accordingly comprises the several steps of matching the recursive filter to CGM data in real-time, then calculating an appropriate insulin dose based on past calibration results. This is to be done in iterative steps so to ensure hypo-glycemia is not induced. The relation of the various steps with respect to each of the others will be based on mathematical constants that are determined with high confidence. These also remain subject to change however in the presence of updated data from the various apparatus embodying features of construction (e.g. CGM and insulin pump measurements). The combinations of elements and arrangement of parts that are adapted to affect such steps will be exemplified in the following detailed disclosure, and the scope of the invention will be indicated in the claims.

### BRIEF DESCRIPTION OF THE DRAWINGS

For a more complete understanding of the invention, reference is made to the following description and accompanying drawings, in which:

FIG. 1 (a) Natural Feedback system in human body where pancreas controls blood sugar during eating and exercise. (b) Closed loop insulin delivery system in diabetic using a control algorithm[8] (developed herein), CGM[9], insulin pump[10] and emergency glucose supply[11];

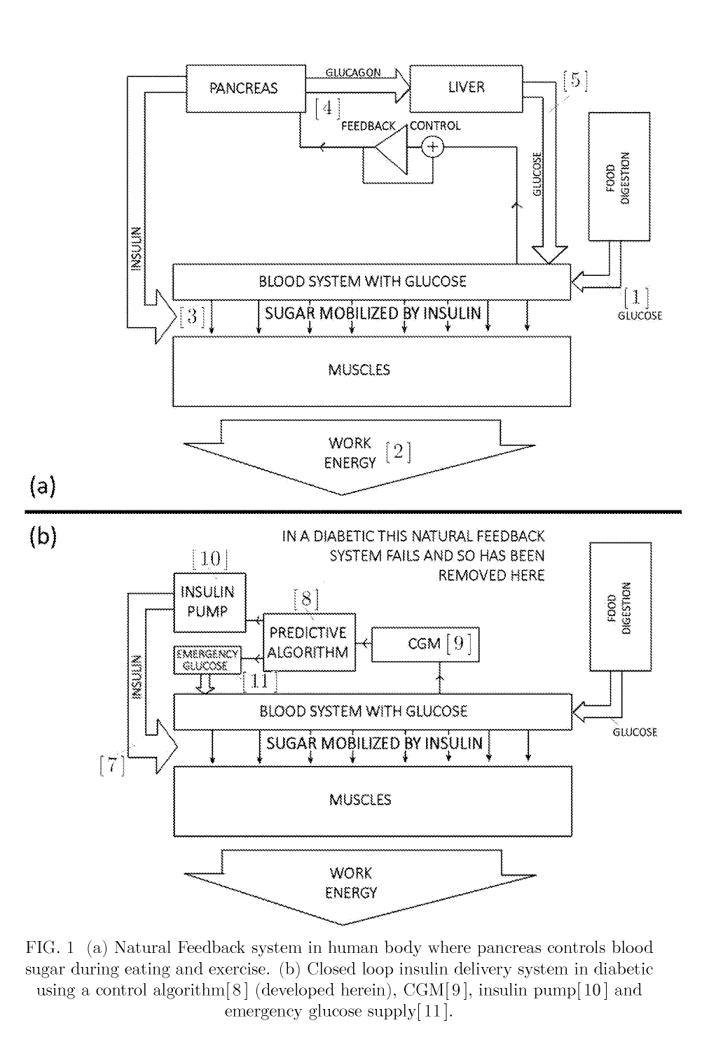

FIG. 2 (a) Left: food absorption rates of fast and slow carbohydrates. Right: fast and slow insulin absorption rates—both graphs from Stahl & Johansson (2009). (b) Example exponential model curves for fast & slow food/insulin impulse response functions. (c) Convolution model of human response to food/insulin, where the patient blood sugar level G(t) is the area overlap between the functions F(t) and H(t′−t);

FIG. 3 (a) 2D landscape visualization of blood glucose change rate for three food events of fast carbohydrate at breakfast and dinner (j=1 & 3) and slow carbohydrate at lunch (j=2). (b) 2D landscape visualization of two insulin events caused by breakfast and lunch-time injections (j=4 & 5);

FIG. 4 Example CGM data and model fit after an insulin event; and

FIG. 5 Example CGM data and model fit after an insulin event.

### DETAILED DESCRIPTION OF THE PREFERRED EMBODIMENTS

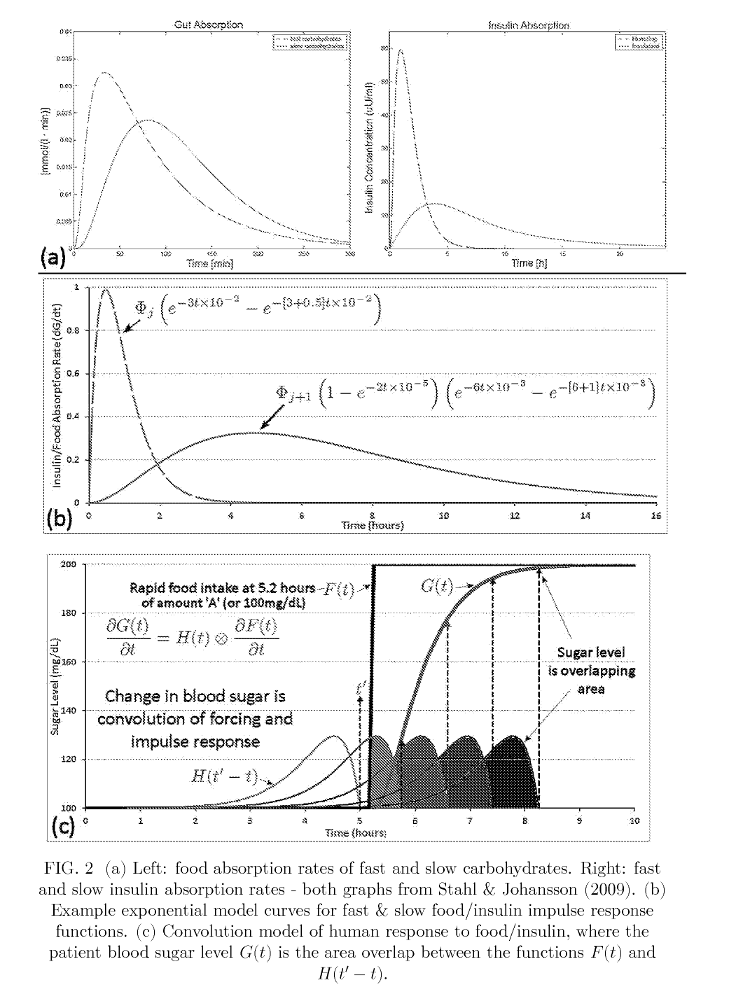

This invention considers that the blood glucose system of the human body responds with an infinite time impulse response function to a forcings. These can come from food digested in the stomach combined with an amount of insulin in the blood and must then be considered in conjunction with the present environment (e.g. the amount of exercise currently undertaken). In a diabetic the time t dependent forcing function F(t) is here not necessarily considered a prediction of where the sugar level will eventually go. Without a natural feedback mechanism, it is instead thought of as where the glucose system is trying to go baring intervention from insulin, more food, increased/decreased exercise or simple changes in environment. In practice the time domain impulse response of the patient glucose change rate due to forcing will not be a constant. As stated it may vary with exercise and environment. This algorithm however initially assumes that the function shape is constant and can be represented by a continuous mathematical distribution H(t). Examples of H(t) for different types of both food and insulin are shown in FIG. 2(a) (from Stahl & Johansson (2009)). This indicates the rate at which either food is converted to blood sugar, or blood sugar converts to energy in the patient. It also illustrates that both food and insulin absorption rates go from zero to a maximum value within around an hour or more, then begin to reduce afterwards. The shapes shown in FIG. 2(a) are hence continuous with only one zero gradient point, meaning that they could be well represented using a function that is the difference of at least two exponentials, as displayed in Eqn. 1:

$$\begin{matrix}H ( t ) = Φ {\prod }_{i = 1}^{N} ( {e}^{- {b}_{i} t} - {e}^{- [ {a}_{i} + {b}_{i} ] t} ) & ( 1 ) \\ {\int }_{0}^{∞} H ( t ) d t = 1 & ( 2 )\end{matrix}$$

aᵢ & bᵢ are positive and real reciprocal time constants that characterize the typical absorption rate of the patient to food or insulin (i.e. aᵢ=1/τᵢ, with τᵢ an absorption time constants, typically around 60 minutes from FIG. 2(a)). Φ is simply a variable that depends on the values of aᵢ & bᵢ making the Eqn. 2 integral equal to one. It helps to initially consider simulation of a situation where a patient eats only a single type of food, with very specific gut absorption properties similar to one of those shown in FIG. 2(a)(left). For the case of this food absorption alone, it is assumed that the function H(t) can be represented using only single values of the aᵢ & bᵢ coefficients (i.e. N=1 in Eqn. 1). The FIG. 2(b) dashed graph shows the model curve from Eqn. 1, with the values aᵢ=1/200 & bᵢ=1/33.33 minutes⁻¹ respectively.

Ideally, a fast acting insulin might be manufactured that has exactly equal time response coefficients, but opposite amplitude to the absorption of the food eaten by the patient. This would mean that the effective food/insulin forcing function F(t) would undergo a positive change for food and an equally negative one for a corresponding insulin injection. The resulting rate of change in blood glucose level would be found by the convolution of the impulse response H(t) with the time differential of the forcing function F(t) as in FIG. 2(c) & Eqn. 3:

$$\frac{\partial G ( t )}{\partial t} = H ( t ) ⊗ \frac{\partial F ( t )}{\partial t} \qquad ( 3 )$$

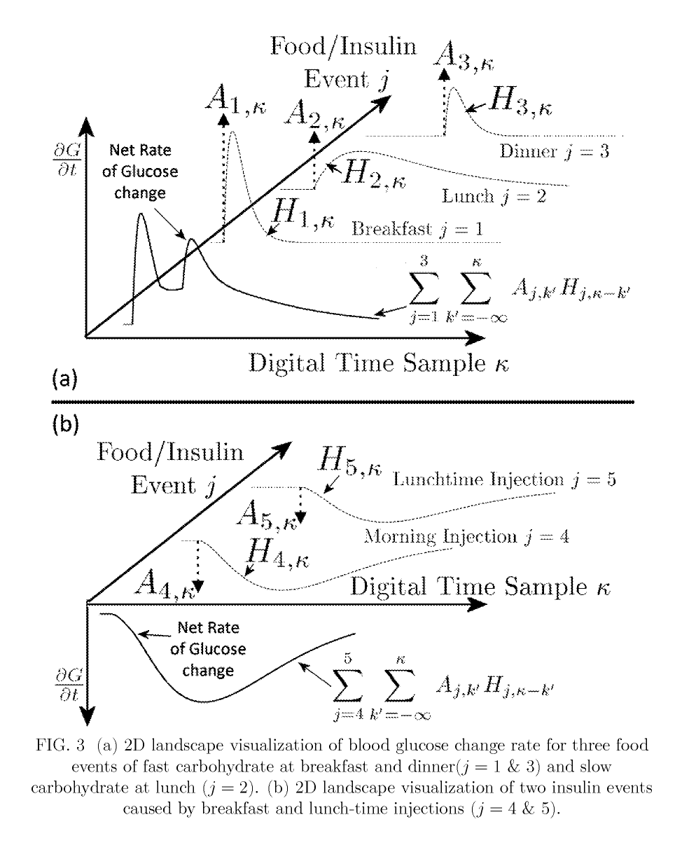

G(t) is therefore the blood glucose level of the patient at time t in mg/dL and the measurement retrieved by a CGM. In the event of an insulin injection or rapid sugar intake at time tₖ, the rate of change ∂F(t)/∂t could be considered a delta Dirac function A.∂(t−tₖ). A is a constant proportional to the amount of insulin injected or food eaten (positive for food as in FIG. 2(c) and negative for insulin as in FIG. 3(b)). In the case of fast and slow acting insulin types, FIG. 2(a) (right) shows the rate of change of blood sugar level ∂G(t)/∂t after an injection (e.g. for Humalog or Insulatard insulin types from Stahl & Johansson (2009)).

The two constant form of this food/insulin impulse response is then found using Eqn. 4 (i.e. dashed curve in FIG. 2(b) with two exponential time constants a and b because N=1 in Eqn. 1):

H(t)=Φe^{-bt}(1−e^{-at}) (4)

Since CGM systems report sugar levels as digital data, it is also convenient to convert continuous functions to the digital time domain for each sample k. Digital time tₖ is then Δt.k where Δt is the sampling interval of a sensor (typically 5 minutes). A digital time domain version of the impulse response function H(t) for sample k is hence given by Eqn. 5.

Hₖ=Φe^{-bΔt.k}(1−e^{-aΔt.k}) (5)

Design of recursive filters for digital data is significantly simplified if performed in the z domain using the unilateral z transform (where z=e^{φ} and φ is an imaginary number). This takes the form of Eqn. 6, which transforms Eqn. 5 to become Eqn. 7:

$$\begin{matrix}H ( z ) = {\sum }_{k = 0}^{∞} {H}_{k} \times {z}^{- k} \\ = Φ {\sum }_{k = 0}^{∞} [ {( {e}^{- b Δ t} {z}^{- 1} )}^{k} - {( {e}^{- ( a + b ) Δ t} {z}^{- 1} )}^{k} ] ( 7 )\end{matrix} \qquad ( 6 )$$

It is important to note that Eqn. 7 represents an infinite geometric summation. This is convenient when considered that a geometric sum of a series hₖ=Φr^{k} can be represented as in Eqn. 8 for n→∞:

$$Φ {\sum }_{k = 0}^{∞} {r}^{k} = \frac{Φ}{1 - r} ∈ \vert r \vert < 1 \qquad ( 8 )$$

In the case of the human food impulse response of Eqn. 7, there are two terms in the z transform summation with common factors r₁=e^{-bΔt}z⁻¹ & r₂=e_{-(a+b)Δt}z⁻¹:

$$\begin{matrix}{H}_{k} = Φ ( {r}_{1}^{k} - {r}_{2}^{k} ) & ( 9 ) \\ \begin{matrix}H ( z ) = Φ ( \frac{1}{1 - {r}_{1}} - \frac{1}{1 - {r}_{2}} ) \\ = Φ ( \frac{1}{1 - {e}^{- b Δ t} {z}^{- 1}} - \frac{1}{1 - {e}^{- ( a + b ) Δ t} {z}^{- 1}} ) ( 11 ) \\ = \frac{Φ ( {e}^{- b Δ t} - {e}^{- ( a + b ) Δ t} ) {z}^{- 1}}{1 - ( {e}^{- b Δ t} + {e}^{- ( a + b ) Δ t} ) {z}^{- 1} + {e}^{- ( a + 2 b ) Δ t} {z}^{- 2}} ( 12 ) \\ = \frac{Φ {c}_{0} {z}^{- 1}}{1 - {c}_{1} {z}^{- 1} + {c}_{2} {z}^{- 2}} ( 13 ) \\ = \frac{Φ {c}_{0}}{z - {c}_{1} + {c}_{2} {z}^{- 1}} ( 14 )\end{matrix} & ( 10 )\end{matrix}$$

The various exponential terms are simplified with the use of the constants c₀ to c₂. As with both the Fourier and Laplace domains, the resultant z domain glucose level G(z) will be the product of the sugar forcing function F(z) and the human response function H (z):

$$\begin{matrix}\begin{matrix}G ( z ) = H ( z ) \times F ( z ) \\ = Φ ( \frac{{c}_{0}}{z - {c}_{1} + {c}_{2} {z}^{- 1}} ) F ( z ) ( 16 )\end{matrix} & ( 15 ) \\ Φ = \frac{1 - {c}_{1} + {c}_{2}}{{c}_{0}} & ( 17 )\end{matrix}$$

where, as before Φ is simply a constant given by Eqn. 17 that ensures the filter has a gain of 1 or that G_{∞}=F_{∞}.

The present invention takes advantage of the property of the z transform, that a general function h(z) when multiplied by z^{u} transforms back to the digital time domain as the same original series but simply advanced by u samples (i.e. hₖ₊ᵤ):

$$\begin{matrix}{\sum }_{k = 0}^{∞} {h}_{k + u} {z}^{- k} = h ( z ) {z}^{u} & ( 18 ) \\ F ( z ) = \frac{G ( z ) z - {c}_{1} G ( z ) + {c}_{2} G ( z ) {z}^{- 1}}{Φ {c}_{0}} & ( 19 ) \\ {F}_{k} = \frac{{G}_{k + 1} - {c}_{1} {G}_{k} + {c}_{2} {G}_{k - 1}}{Φ {c}_{0}} & ( 20 ) \\ {F}_{k - 1} = \frac{{G}_{k} - {c}_{1} {G}_{k - 1} + {c}_{2} {G}_{k - 2}}{Φ {c}_{0}} & ( 21 )\end{matrix}$$

Eqn. 16 can be re-arranged to become Eqn. 19, which gives the sugar forcing function F(z) in terms of glucose level G(z). It is then simple to transform this into the digital time domain to give the recursive relationship of Eqn. 20 or 21.

This analysis is a simplified example that assumes the possibility of a constant and matched food/insulin time response, without instrument noise present. In such a case it would be straightforward to directly use CGM measurements in determining the ultimate sugar level (Fₖ₋₁≈F_{∞}) that the patient is being forced to from Eqn. 21. In theory after eating food, with a perfect prediction of where the patients sugar level is being forced to and knowledge of the effective ‘gain’ g of the insulin, a required insulin dose I is calculated as I=(F_{∞}−F_{T})/g. This would precisely counter the sugar forcing of the food and leave the patient with a final target sugar level of F_{T} (typically desired to be around 100 mg/dL). The insulin gain ‘g’, in sugar units of mg/dL per ml of insulin injected, would be fairly constant but could also depend on environmental conditions to some extent. This example therefore shows how a recursive filter can be used to predict where a physically understood system is being forced to without the presence of instrument noise.

Building on the instrumental noise free premise given above, further variation and dimension will now be added to the model. This shall make it able to accurately represent real world changes to a patient glucose level when eating food and injecting artificial insulin during a typical day. In addition, actual data from a currently available CGM sensor must be used as the only input for the algorithm's design of insulin delivery amount.

To do this a discrete ‘food or insulin event’ of type j is considered as in FIG. 3. Each event j has specific time constant characteristics aᵢⱼ & bᵢⱼ, such that its time response is given by Eqn. 22 with normalized characteristics of Eqn. 23:

$$\begin{matrix}{H}_{j , k} = {Φ}_{j} {\prod }_{i = 1}^{N} ( {e}^{- {b}_{ij} Δ t . k} - {e}^{- [ {a}_{ij} + {b}_{ij} ] Δ t . k} ) & ( 22 ) \\ Δ t {\sum }_{k = 0}^{∞} {H}_{j , k} = 1 & ( 23 )\end{matrix}$$

An event j could be eating breakfast or lunch, then injecting insulin of various types using a syringe or insulin pump (i.e. a bolus). It is expected that there could be dozens (or a number M) of such events during the course of the previous 24 hours. To graphically visualize this, the single dimension of FIG. 2 is expanded upon to two as in FIG. 3. Here as before there is the dimension of time κ (temporary version of k) and also the j dimension of different food/insulin types or events. The past day can therefore be represented as a series of food/insulin events occurring at digital sample time k_{f}^{j}/k_{I}^{j}, of type j and amplitude A_{j,k}_{_{f}}_{^{j}}/A_{j,k}_{_{I}}_{^{j}}(in mg/dL/minute). The 2D landscape displayed in FIG. 3(a) hence represents breakfast, lunch and dinner. FIG. 3(b) then shows the corresponding breakfast and lunch-time insulin injections which are used to regulate the change in blood sugar. The glucose level of the patient at time sample k is then given by Eqn. 24, as the summation and convolution of all the A_{j,k} & H_{j,k} values over the previous day. FIG. 3 also shows the net rate of change from all food and insulin events j respectively along the κ or time axis.

$$\begin{matrix}{G}_{k} = Δ t {\sum }_{{k}^{′} = 0}^{k} {\sum }_{j = 1}^{M} {\sum }_{κ = - ∞}^{{k}^{′}} {A}_{j , κ} {H}_{j , {k}^{′} - κ} & ( 24 ) \\ {F}_{k - 1} = Δ t {\sum }_{{k}^{′} = 0}^{k - 1} {\sum }_{j = 1}^{M} {A}_{j , {k}^{′}} & ( 25 )\end{matrix}$$

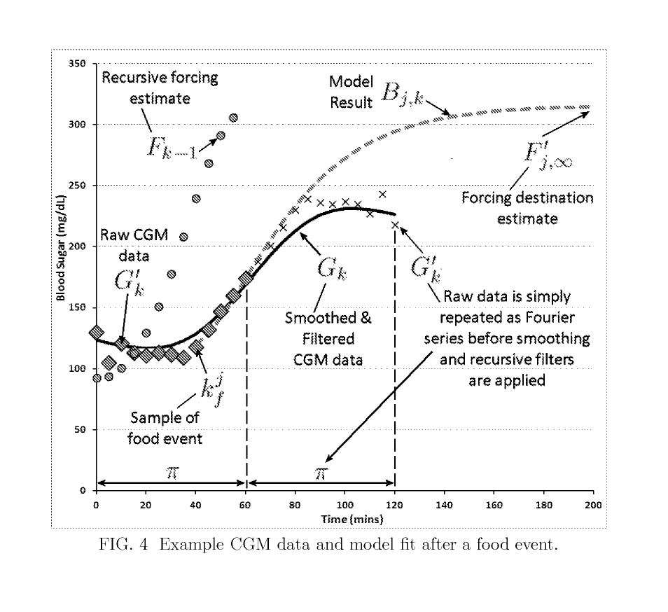

For effective closed loop insulin delivery, a reliable estimate is needed of what blood sugar the patient is currently being forced to. This is given as Fₖ₋₁ in Eqn. 25 and is the summation of all the forcing amplitudes A_{j,k} which have occurred recently. This requires de-convolution of the delay effects in CGM data from stomach/insulin absorption that are characterized here by exponential functions H_{j,k} from Eqn. 22. Practically this will be done using Eqn. 21 and by making time specific estimates of the forcing amplitudes A incurred by the patient throughout the day. The required insulin dose I to stabilize the patient at the target sugar level F_{T} after eating food j can then be calculated simply as I_{j+1,k}=(Fₖ₋₁−F_{T})/g≈(F_{j,∞}−F_{T})/g (see example later in FIG. 4).

The following seven paragraphs explain details of how estimates of F_{3,∞} (and hence sum of amplitudes A) are made based on CGM data alone in a practical manner, using the recursive relationship defined in Eqn. 21 as a pre-estimate of the event occurring (i.e. either a food or Insulin intake). To do this however, there are two important practical issues that must first be addressed and which stem from the nature of the human time response to food & insulin (see FIG. 2(a) & (b) and Eqn. 1). First note that H(t) has an initial start value of zero and as shown in Eqn. 21, this causes the estimate of forcing destination Fₖ₋₁ to require use of CGM measurements made at times tₖ₋₂, tₖ₋₁ & tₖ (i.e. past, present and future). Mathematically this also means that the recursive filter estimate will need to lag one sample behind the latest CGM measure. Second, the low initial values of the H(t) function could result in Eqn. 21 amplifying any CGM instrumental noise to unstable levels. In order to optimize diabetic closed loop control, the following processing of raw CGM data is necessary to counter these two effects.

Raw CGM data is defined here as G′ₖ, an example of which is displayed in FIG. 4 as diamond shaped data points. Only measurements up to sample k will exist, now in practice with instrument noise being present. Eqn. 21 requires low noise measurements of glucose levels up to and beyond sample k−1. The noted smooth exponential nature of the human food/insulin impulse response (FIG. 2) means that it can also be represented using a Fourier series with minimal coefficients (or low frequency information). Hence a simple low pass filter can be applied to the raw data G′ₖ to remove instrument noise (which as discussed could make the Eqn. 21 result unstable). This low time frequency nature of human response then means that it can be well represented as a repeating Fourier series, with only a few low order coefficients. This also is shown as the Gₖ function in FIG. 4, repeating after one π half cycle. Such low pass filtering can of course be done in several ways depending on processing capabilities (e.g. time convolution with a Gaussian filter or simple attenuation in the Fourier domain as was used to give the solid curve function Gₖ in FIG. 4). Since the low pass filtered result Gₖ is considered a repeating Fourier series, it therefore allows a pre-estimate of the value to occur after sample k. This is convenient in the use of low pass filters which typically benefit from the presence of samples beyond that at the time desired (e.g. at k−1, see FIG. 4 where Gₖ is assumed to repeat as the solid smooth curve after the first π half cycle).

Now the low pass filtered CGM result Gₖ (created from the raw G′ₖ samples as in FIG. 4) can be used in the recursive relationship of Eqn. 21 to make an initial estimate of Fₖ₋₁. This hence predicts where the patients' blood sugar is currently being forced to and is temporarily considered representative (i.e. that the patient only eats one type of simple food and has perfect insulin that exactly counters its effects). Using pre-determined optimum time constants a & b, this estimate of where the patient is being forced to is gained from Eqn. 21 and shown as circles in FIG. 4. In order to decide if a food event has occurred with confidence, this estimate is compared to the last recursive result, telling of where the patient was last being forced to (i.e. compare Fₖ₋₁ to Fₖ₋₂≈F_{j,∞}, looking for Fₖ₋₁−Fₖ₋₂ to exceed a threshold value +ΔF and k_{f}^{j} becomes the sample at which the food event was decided to have occurred). As an example, in FIG. 4 it is assumed that the algorithm has been initiated with a new unknown patient, where 30 minutes of CGM data has been taken up to this point (i.e. k must be at least 6). In the start case only, F_{-1,∞} is set to Gₖ (from Eqn. 21 assuming that there are no food or insulin forcings currently in the patients system). In the event that F_{j,k}_{_{f}}_{^{j}}−F_{j-1,∞}≧ΔF with new CGM data, the algorithm therefore decides that a food event has just occurred (where ΔF=20 mg/dL in this example but also may vary with different patients). FIG. 4 shows this, with results from a diabetic patient where a meal has recently been eaten. Thus the sugar level begins to rise and the curvature of Gₖ also increases, causing a jump in the recursive result Fₖ₋₁ (circles). The estimate of the food event amplitude is then found simply as A_{j,k}_{_{f}}_{^{j}}=F_{j,k}_{_{f}}_{^{j}}−F_{j-1,∞} (where the trigger sample k_{f}^{f} is typically k−1 but can be chosen from the 3 samples between k−3 & k−1 so to maximize the amplitude of A_{j,k}_{_{f}}_{^{j}}).

Since the time of the food event is unknown but certainly before to tₖ_{_{f}}_{^{j}}, a digital phase delay Δkⱼ is used to represent how long ago food was eaten (typically chosen from 0→9 or 0-45 minutes). The food type is also unknown, so an estimate must be made of its time absorption characteristics. This uses an iterative fit to the actual CGM data (i.e. decide on some optimal values of a_{1,j}, b_{1,j} & Δk₃ in Eqns. 26 and 27, so to best match the data occurring after sample k_{f}^{j}−Δkⱼ).

$$\begin{matrix}{H}_{j , k} = Φ {e}^{- {b}_{1 , j} Δ t . k} ( 1 - {e}^{- {a}_{1 , j} Δ t . k} ) & ( 26 ) \\ {S}_{j , κ} = {\sum }_{k = {k}_{f}^{j}}^{κ} {A}_{j , {k}_{f}^{j}} \times {δ}_{( k , {k}_{f}^{j} - Δ {k}_{j} )} & ( 27 ) \\ {B}_{j , k} = Φ {c}_{0} {S}_{j , k - 1} + {c}_{1} {B}_{j , k - 1} - {c}_{2} {B}_{j , k - 2} & ( 28 ) \\ {Ψ}_{j , k} = {\sum }_{κ = {k}_{f}^{j} - Δ {k}_{j}}^{k} {( {G}_{κ}^{′} - {B}_{j , κ} )}^{2} & ( 29 )\end{matrix}$$

The Kronecker delta function δ_{(k,k}_{_{f}}_{^{j}}_{-Δk}_{ⱼ}₎ in Eqn. 27 is zero everywhere except for at k=k_{f}^{j}−Δkⱼ, where it has a value of one. Again note that the apostrophe on G′ₖ in Eqn. 29 indicates that it is the raw unfiltered CGM result, with a preserved temporal frequency response so to best match the model being iterated. This model estimate of the raw data G′ₖ is therefore generated with iterative estimates of forcing F, by simple re-arrangement of Eqn. 21 to give the recursive model result B_{j,k} for food event j (Eqn. 28). A gradient descent algorithm as described by Matthews (2009) is then used to find the optimum values of a_{1,j}, b_{1,j} & Δkⱼ that minimize the residue result Ψ_{j,k} (Eqn. 29). An example of the optimal model fit B_{j,k} is also shown as the dashed line in FIG. 4. The next requirement is now to estimate the amount of insulin needed to counter this detected food event, which requires two more steps:

G′ₖ=mⱼB_{j,k}+Cⱼ (30)

F′_{j,∞}=?mⱼ×A_{j,k}_{_{f}}_{^{j}}+Cⱼ (31)

I_{j,k}=(F′_{j,∞}−F_{T})/g (32)

First the model result B_{j,k} is linearly regressed against the actual data G′ₖ as in Eqn. 30, to give slope and offset values mⱼ & Cⱼ (typically mⱼ≈1 & Cⱼ=Gₖ_{_{f}}_{^{j}}_{-Δk}_{ⱼ}). Then the new and more accurate estimate of the forcing destination F′_{j,∞} (Eqn. 31) is again compared to the threshold sugar level F_{T} (nominally 100 mg/dL). The variable g must be a conservative estimate of insulin gain, so to calculate an effective but safe dose I_{j,k} from Eqn. 32 to then be injected from the insulin pump[10]. As the next sample G′ₖ₊₁ arrives from the CGM, the value of A_{j,k}_{_{f}}_{^{j}}, is recalculated. If it and Fₖ₋₁ have increased in value, they are updated and the gradient descent fit of Eqns. 26 to 29 is repeated, this time with the extra CGM data point. In the event that the resulting insulin dose I_{j,k+1}>I_{j,k}, then the additional amount I_{j,k+1}−I_{j,k} is simply then injected from the pump[10].

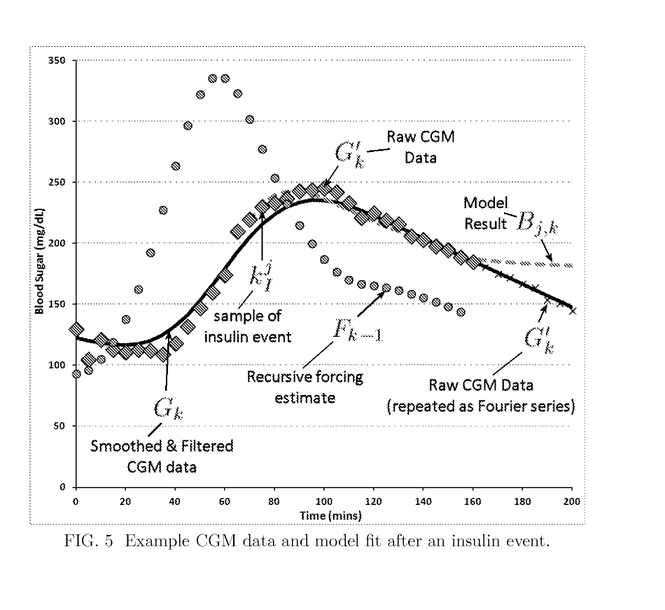

The recursive/iterative process continues and the model results are updated and stored to estimate the amount of both food and insulin in the patients system as more data arrives. Eventually the patient blood glucose G′ₖ ceases to rise and the continually calculated forcing values Fₖ₋₁ then conversely drop a threshold value ΔF below the model destination (i.e. Fₖ₋₁−F′_{j,∞}≦ΔF). This indicates that an insulin event has occurred. FIG. 5 shows an example of this with further CGM data sampled beyond that in FIG. 4, where an Insulin event is found to have occurred. Similar to the food event discussed above, the amplitude of this event A_{j,k}_{_{I}}_{^{j}} is calculated as F_{j,k}_{_{I}}_{^{j}}−F_{j-1,∞} (where like k_{f}^{j}, k_{I}^{j} is usually k−1 but can take a value between k−3 and k−1 if it maximizes the absolute value of A_{j,k}_{_{I}}_{^{j}}). As discussed earlier, detection of both food and insulin events is initiated using the simplistic assumption that both have time responses that are equal in shape but opposite in sign. (i.e. since insulin events lower blood sugar levels in the patient, their amplitudes are negative). However as also mentioned the algorithm must operate with the conservative expectation that a restoring injection from a sugar reservoir is to be used only in an emergency. In general the type of insulin being injected will be more of a constant than the type or frequency of food eaten. In order to proceed with extra caution, it is therefore optimum to use an extra term in Eqn. 1 for a representation of the insulin impulse response. This also helps to recreate the inflection points often observed as part of the insulin time response, in addition to allowing simulation of slower acting insulin (e.g. see solid curve of FIG. 2(b)). The following Eqns. 33 to 48 are hence simply slightly higher order versions of those presented in Eqns. 26 to 29, with three rather than two exponential terms:

$$\begin{matrix}{H}_{j , k} = {Φ}_{j} ( 1 - {e}^{- {a}_{2 , j} Δ t . k} ) ( {e}^{- {b}_{1 , j}} - {e}^{- [ {a}_{1 , j} + {b}_{1 , j} ] Δ t . k} ) & ( 33 ) \\ p = - ( {a}_{2 , j} + {b}_{1 , j} ) Δ t . k & ( 34 ) \\ q = - ( {a}_{1 , j} + {b}_{1 , j} ) Δ t . k & ( 35 ) \\ r = - ( {a}_{2 , j} ++ {a}_{1 , j} + {b}_{1 , j} ) Δ t . k & ( 36 ) \\ s = - ( {b}_{1 , j} ) Δ t . k & ( 37 ) \\ {d}_{0} = {e}^{r} + {e}^{s} - {e}^{p} - {e}^{q} & ( 38 ) \\ {d}_{1} = 2 ( {e}^{p + q} - {e}^{s + r} ) & ( 39 ) \\ {d}_{2} = {e}^{s + q + r} + {e}^{p + s + r} - {e}^{p + s + q} - {e}^{p + q + r} & ( 40 ) \\ {d}_{3} = {e}^{p} + {e}^{q} + {e}^{r} + {e}^{s} & ( 41 ) \\ {d}_{4} = {e}^{p + q} + {e}^{p + r} + {e}^{p + s} + {e}^{q + r} + {e}^{q + s} + {e}^{r + s} & ( 42 ) \\ {d}_{5} = {e}^{p + q + r} + {e}^{s + q + r} + {e}^{p + s + r} + {e}^{p + s + q} & ( 43 ) \\ {d}_{6} = {e}^{p + q + r + s} & ( 44 ) \\ {Φ}_{j} = \frac{1 - {d}_{3} + {d}_{4} - {d}_{5} + {d}_{6}}{{d}_{0} + {d}_{1} + {d}_{2}} & ( 45 ) \\ {S}_{j , κ} = {\sum }_{k = {k}_{I}^{j}}^{κ} {A}_{j , {k}_{I}^{j}} \times {δ}_{( k , {k}_{I}^{j} - Δ {k}_{j} )} & ( 46 ) \\ {B}_{j , k} = {Φ}_{j} ( {d}_{0} {S}_{j , k - 1} + {d}_{1} {S}_{j , k - 2} + {d}_{2} {S}_{j , k - 3} ) + {d}_{3} {B}_{j , k - 1} - {d}_{4} {B}_{j , k - 2} + {d}_{5} {B}_{j , k - 3} - {d}_{6} {B}_{j , k - 4} & ( 47 ) \\ {Ψ}_{j , k} = {\sum }_{κ = {k}_{f}^{j} - Δ {k}_{j}}^{k} {( {G}_{κ}^{′} - {B}_{j , κ} )}^{2} & ( 48 )\end{matrix}$$

Again a Kronecker delta function δ_{(k,k}_{_{I}}_{^{j}}_{-Δk}_{ⱼ}₎ is zero everywhere except for at k=K_{I}^{j}−Δkⱼ where it has a value of one (Eqn. 46) and as before the apostrophe on the G′ₖ indicates that is the raw unfiltered CGM result (preserving the true frequency response). The model estimate of the raw data G′ₖ is now generated as the recursive result B_{j,k} for insulin event j from Eqn. 47 (i.e. using iterated model estimates S_{j,k} of net forcing F). Optimum values of a_{1,j}, b_{1,j}, a_{2,j} & Δkⱼ are also determined from a slightly higher order gradient descent algorithm so to again minimize the residue result ψ_{j,k} in Eqn. 48 (see Matthews (2009)). Greater confidence is again obtained by re-performing the regression of Eqns. 30 and 31 (where as before mⱼ≈1 but now Cⱼ=Gₖ_{_{I}}_{^{j}}_{-Δk}_{ⱼ}₎. Such an example of an insulin event optimal model fit B_{j,k} is shown in FIG. 5 as the dashed curve occurring after a time of 160 minutes.

This document details the basis behind use of a recursive filter to model, fit and predict a diabetic response to either food or insulin intake in real-time. The algorithm allows a computer to design an insulin pump dose to safely restore blood glucose levels to normal, through what is typically known as a closed loop insulin control system. The process has been demonstrated using computer code written in the IDL language.

It will thus be seen that the objects set forth above, among those made apparent from the preceding description, are efficiently attained and, because certain changes may be made in carrying out the above method and in the construction(s) set forth without departing from the spirit and scope of the invention, it is intended that all matter contained in the above description and shown in the accompanying drawings shall be interpreted as illustrative and not in a limiting sense.

It is also to be understood that the following claims are intended to cover all of the generic and specific features of the invention herein described and all statements of the scope of the invention which, as a matter of language, might be said to fall there between.

What is claimed:

1. A system for predicting the blood glucose level to which a diabetic patient is being forced after food and insulin intake comprising:
A continuous glucose monitoring (CGM) device transmitting blood glucose measurements as digital data and,
a blood glucose predictor algorithm in a computer worn by the patient that receives this digital data, and based on it alone predicts the ultimate sugar level to which the patient is currently being forced, wherein the blood glucose estimate includes a low pass filter for removing instrumental noise prior to use in recursive equations, and
these recursive equations are designed in the z domain based on exponential equation fits to observed blood glucose change rates after eating and insulin injections and,
two separate equations of increasing complexity are used for food and insulin time response respectively, with the exponential coefficients involved chosen based on gradient descent iterative fits to recent CGM data and,
the predicted level enables calculation of a conservative insulin bolus dose to be delivered if needed by an insulin pump, so to restore blood glucose levels to normal.

2. The method of claim 1 wherein
the human food and insulin time response filters are compromised of the difference between exponential functions in the z domain with number of terms n−1 and n, where n is a positive integer.

3. The method of claim 1 wherein
The separate food and insulin time response filters have n−1 and an n exponential terms respectively, where n is a positive integer.

4. The method of claim 3 wherein
n is the number 3.

5. The method of claim 3 wherein
the food and insulin time response filters are infinite response curves with a single zero gradient point that both decrease monotonically over time.

6. The method of claim 3 wherein
the insulin time response filter curves is sufficiently sophisticated so to allow safer insulin dose calculation with an inflection point in its shape prior to a zero gradient peak.

7. The method of claim 3 wherein
insulin amplitude response is characterized by a single variable called insulin gain.

8. The method of claim 1 wherein
after the recursive prediction of food or insulin intake and prior to activating the insulin pump, additional confidence is gained using an iterative gradient descent fit of the relevant filter equation to raw un-filtered CGM data.

9. The method of claim 1 wherein
the patient uses a closed loop insulin control system consisting of a CGM, insulin pump, computer processor and emergency glucose injection reservoir.

10. A filtering system comprising:
standard and appropriate low pass filter applied to CGM data, and Fourier series repeating of existing data to estimate results beyond sample k−1, and separate recursive exponential filters for de-convolution of food and insulin responses, with n−1 and n terms respectively.

11. The method of claim 13 wherein
n is a positive integer.

12. The method of claim 11 wherein
n is the number 3.

13. A method of filtering CGM data and recursively predicting the blood glucose level to which a diabetic is being forced comprising the steps of:
receiving digital CGM data for analysis by a computer processor;
low pass filtering such data prior to use in recursive equation;
producing recursive prediction of destination blood sugar level;
use of a threshold of second to last, compared to last prediction, to detect either a food or insulin event;
gradient descent iterative fit of either food or insulin equations to raw unfiltered CGM data;
in the event of confidence in the predicted level, insulin pump bolus dose is calculated based on conservative estimate of insulin gain;
recursive and iteration process repeats upon arrival of new CGM data (typically every 5 minutes).
